(19) United States
(12) Patent Application Publication
(10) Pub. No.: US 2012/0064053 A1
(43) Pub. Date: Mar. 15, 2012

(54) Baby Lotion

(76) Inventor: H. Lance Johnson, Charlotte, NC (US)

(21) Appl. No.: 12/826,452

(22) Filed: Sep. 12, 2010

(51) Int. Cl. A61K 8/66; A61Q 19/00

(52) U.S. Cl. 424/94.1

(57) ABSTRACT

The skin/body lotion composition of the present invention utilizes all natural plant derived components as a method for conditioning and moisturizing the skin. The invention provides good moisturizing and gives a smooth, soft touch feel upon conditioning of the skin, leaving no oily or greasy residue on the skin and is excellent in stability.

Product: Baby Lotion

| Chemir | | | % by |
|---|---|---|---|
| ID# | Phase | Ingredients | WT |
| N/A | A/D | Deionized Water | 72.6900 |
| 590555 | C | Caprylic/Capric Triglyceride | 7.0000 |
| 591439 | C | Sunflower seed oil | 3.5000 |
| 581483 | C | Butyrospermum Parkii (Shea Butter) | 4.0000 |
| 590598 | C | jojoba seed oil | 2.7000 |
| 598063 | C | stearic acid | 3.0000 |
| 583157 | C | Cetearyl Alcohol and Cetearyl Glucoside | 2.5000 |
| 590092 | C | Glyceryl Stearate | 1.2000 |
| 582818 | B | Xanthan gum | 0.9000 |
| 590055 | F | Biovert Substrate (glucose) | 1.0000 |
| 590054 | F | Biover Enzyme (Glucose oxidase/ | 0.0500 |
| | | lactoperoxidase | |
| 591797 | E | Fragrance #74962 Milk & Chamomile | 1.0000 |
| 591514 | D | Caramel AP100 | 0.0600 |
| 590284 | E | Tocopheryl Acetate (Vitamin E) | 0.1000 |
| 591607 | E | Aloe Vera Barbadensis Leaf Juice | 0.2500 |
| 591515 | G | Potassium Sorbate | 0.0500 |
| | | TOTAL: | 100.0000 |

I claim:

1. A one-part all natural baby lotion composition comprising the following ingredients (A) to (P): (A) from greater than 50% deionized water; (B) 25 to 50% caprylic/capric triglyceride; (C) 1 to 5% of butyrospermum parkii; (D) 1 to 5% sunflower seed oil; (E) 1 to 5% stearic acid; (F) 1 to 5% jojoba oil; (G) 1 to 5% cetearyl alcohol and cetearyl glucoside; (H) 1 to 5% glyceryl stearate; (I) 1 to 5% biovert substrate; (J) 1 to 5% natural fragrance #74962; (K) 0.1 to 1% xanthum gum; (L) 0.1 to 1% aloe vera barbadensis leaf juice; (M) 0.1 to 1% tocopheryl acetate; (N) less than 0.1% natural caramel color; (O) less than 0.1% potassium sorbate; and (P) less than 0.1% biovert enzyme.
